(19) United States
(12) Patent Application Publication
(10) Pub. No.: US 2021/0101953 A1
(43) Pub. Date: Apr. 8, 2021

(54) Human Interleukin-2 and Anti-human Epithelial Cell Adhesion Molecule Single-Chain Antibody Fusion Protein and Application Thereof

(71) Applicant: ZHEJIANG K2 ONCOLOGY CO., LTD., Huzhou (CN)

(72) Inventors: Zhijian SUN, Huzhou (CN); Ping KANG, Huzhou (CN); Minghao ZHANG, Huzhou (CN)

(21) Appl. No.: 17/065,052

(22) Filed: Oct. 7, 2020

Related U.S. Application Data
(63) Continuation of application No. PCT/CN2019/081852, filed on Apr. 9, 2019.

(30) Foreign Application Priority Data
Apr. 10, 2018 (CN) 201810317694.X

(51) Int. Cl. C07K 14/55; C07K 16/30

(52) U.S. Cl. CPC C07K 14/55; C07K 2317/622; C07K 2319/00; C07K 16/30

(57) ABSTRACT

A fusion protein, which is characterized in that the fusion protein is composed by interleukin-2 (IL-2) being fused with and linked to an anti-human epithelial cell adhesion molecule (EpCAM) single-chain antibody. Further provided by the present solution is a use of the fusion protein in the preparation of a drug for treating tumors.

### CROSS REFERENCE TO RELATED APPLICATIONS

The present application is a continuation application under 35 U.S.C. § 120 of PCT application No. PCT/CN2019/081852 filed on Apr. 9, 2019, which claims priority to Chinese Application No.: CN 201810317694.X, filed on Apr. 10, 2018, the contents of each of which are incorporated by reference in their entirety for all purposes.

The material in the ASCII text file inno_10256_20201113_sequence_listing_amend, filed with the Preliminary Amendment via EFS-web, created on Oct. 23, 2020, having the size of 33 KB, is incorporated by reference in the specification for all purposes.

### FIELD OF THE INVENTION

This disclosure relates to genetic engineering, medicines and biomedical treatment, and specifically relates to a fusion protein of a human interleukin (IL-2) and an anti-human epithelial cell adhesion molecule (EpCAM) single chain antibody, a nucleic acid molecule encoding the fusion protein, a pharmaceutical composition and a kit comprising the fusion protein, and use of the fusion protein in treating tumor diseases.

### BACKGROUND

Human interleukin-2 (IL-2) is a cytokine secreted by auxiliary T lymphocytes, is mainly produced by activated Th1 cells, plays an important role in the normal regulation and defense mechanism of the body immune system, and plays a core role in regulation of specific cellular immunity reaction. IL-2 does not directly kill tumor cells, but indirectly exerts an anti-tumor effect by stimulating and activating effector cells. There are three main types of effector cells participating in tumor immunization, including T lymphocytes, natural killer cells, and macrophages, wherein cytotoxic T cells (CTL cells) play a major role. IL-2 is bonded with IL-2R on the surface of a target cell in vivo to trigger a signal cascade reaction at the downstream to produce a cell proliferation effect mediated by IL-2, which can stimulate T cell growth, division and differentiation to form CTL cells and enhance the cytotoxic activity thereof, and can promote the proliferation and cytolysis effect of immature CTL cells. Meanwhile, IL-2 can further release the immunosuppression state of tumor infiltrating lymphocytes (TIL cells) and enhance the immune function of the TIL cells, induce proliferation of lymphokine activated killer cells (LAK cells) and promote the activity of the LAK cells, promote NK cell activation, differentiation and proliferation, maintain long-term growth of the NK cells and enhance the activity of the NK cells, stimulate B lymphocytes proliferation and to generate immune globulins, and stimulate the cytotoxic activity of macrophages; besides, IL-2 can further induce the generation of other cytokines (such as TNF-α, IFN-γ, etc.) or induce the expression of cytokine receptors, thereby exerting host anti-tumor immunity effect.

Interleukin-2 drugs (such as Proleukin) are used to treat metastasize lung cancer, advanced melanoma, some lymphomas, and leukemia. However, interleukin-2 under a physiological condition achieves a biological function when it is locally high in concentration. Under a systemic administration condition, an effective local drug concentration cannot be achieved, and it is easy to cause some systemic toxic and side effects, but the concentration at a tumor site is not high enough to reach the concentration that steadily results in drug effects, thus the therapeutic concentration space of the drug is limited. Antibody-coupled or fused interleukin-2 can achieve a higher local concentration of interleukin-2 by virtue of specific targets, thereby reducing the toxicity and improving the drug efficacy.

The epithelial cell adhesion molecule (epithelial cell adhesion molecule, EpCAM) is the first type I transmembrane glycoprotein found in the colon cancer; the epithelial cell adhesion molecule has a molecular mass of 40 kDa and is composed of 314 amino acid residues; and the molecular structure comprises an extracellular domain (EpEX), a single transmembrane domain and an intracellular domain (EpICD), which can mediate adhesion between non-Ca2+-dependent homologous cells. EpCAM is widely expressed in normal tissues of epithelial origin, and is overexpressed to varying degrees in a variety of epithelial cancer tissues; and it has been confirmed by a plurality of studies that EpCAM promotes cell cycle and cell proliferation and is associated with tumorigenesis, progression, invasion, metastasis and the like. In recent years, EpCAM has been confirmed by a plurality of studies as a marker of circulating tumor cells and tumor stem cells.

So far, there are five reported drugs of antibody-coupled or fused interleukin-2 in clinical trials, for example, EMD 273066 (tucotuzumab celmoleukin; huKS-IL2) (US 2007/0036752 A1) is an anti-EpCAM humanized monoclonal antibody-fused interleukin-2 for triggering an immune response. This drug is currently in phase I/II clinical development of the ovarian cancer and the small cell lung cancer SCLC. EMD 273066 is a complete humanized IgG4 antibody-fused interleukin-2 (IgG4-(EU)-(Lys to Ala)-IL-2) with a molecular weight of 167.5 Kda. Although the complete antibody structure provides relatively good stability, it also generates greater steric hindrance, which limits more flexible binding of IL-2. Although this antibody concentrates the level of IL-2 around a targeting molecule, it activates the surrounding immune cells. However, due to steric hindrance, the recognition and signal transduction of target cell membrane surface proteins and immune cell membrane surface proteins are limited. It has been proved by facts that although the drug concentration is reduced and the toxic and side effects are reduced, the curative effect of the drug is not significantly improved. The injection concentration of traditional IL-2 drug treatment is 100 Mio IU/m2, and injection is performed once a week. Antibody-coupled or fused IL-2 is injected once every 20 to 28 days at a concentration of 67.5-110 Mio IU IL-2/m2.

In addition, due to the problem about drug entry in solid tumors, immune cells and macromolecular drugs are both difficult to enter in the tumor, which limits the tumor-killing effect of macromolecular antibody drugs and tumor immune cells. To solve this problem, the present solution adopts a single-chain antibody instead of a complete double-chain plus light-chain complete antibody structure, the molecular weight is reduced from 167.5 Kda to 46 Kda, the efficiency of protein drugs entering the tumor is improved, and by coupling with the targeting molecules on the tumor surface and the IL2R on immune cells, the immune cells are promoted to infiltrate into the tumor.

### SUMMARY

In view of the above-mentioned defects and deficiencies in the prior art, the present solution adopts an anti-EpCAM single-chain antibody-coupled genetically engineered interleukin-2, the molecular weight is about 46 Kda, and the linking fragment between the single-chain antibody and interleukin-2 is lengthened to make the molecular spatial structure be more flexible. The present solution designs a group of fusion protein molecules of anti-EpCAM single-chain antibodies and IL2, and verifies that the fusion protein has quite good biological functions. Smaller molecules shorten the distance between target molecule expressing cells and immune cells. The recognition and signal transduction of immune cells and target molecule expressing cells are promoted. Smaller molecules can achieve higher potency and have higher affinity.

The specific technical scheme of the present disclosure is as follows:

The present solution provides a fusion protein which forms EPIL2 by fusion and linkage of interleukin-2 (IL-2) and an anti-human epithelial cell adhesion molecule (EpCAM) single-chain antibody.

The interleukin-2 (IL-2) may be selected from a wild-type IL-2 or a genetically engineered IL-2, preferably the genetically engineered IL-2; the amino acid sequence of the wild-type IL-2 is shown in SEQ ID No.2; the genetically engineered IL-2 has at least 3 amino acid residue substitutions; with reference to the amino acid sequence SEQ ID No.2 of the wild-type IL-2, the amino acid residue substitutions are preferably C125S, V69A, I128T, and/or Q74P; and more preferably, the amino acid sequence of the genetically engineered IL-2 is shown in SEQ ID No.4, 6 or 8.

The amino acid sequence of the anti-human epithelial cell adhesion molecule (EpCAM) single-chain antibody is shown in SEQ ID No.11 or 13.

Preferably, the amino acid sequence of the fusion protein is shown in SEQ ID No.15 or 17.

The present disclosure provides a separated nucleic acid molecule encoding the above-mentioned fusion protein, and the nucleic acid molecule preferably has a nucleotide sequence shown in SEQ ID No.16 or 18.

The present disclosure provides a vector comprising the above-mentioned separated nucleic acid molecule.

The present disclosure provides a cell comprising the fusion protein EPIL2 or the nucleic acid molecule encoding the fusion protein and is used to produce the fusion protein; and the cell is selected from non-human mammalian cells, preferably CHO and HEK293 cells.

The present disclosure provides application of the fusion protein EPIL2 and/or the nucleic acid molecule encoding the fusion protein in the preparation of a pharmaceutical composition or an in-vitro diagnostic kit; the pharmaceutical composition is preferably a pharmaceutical composition for treating a tumor; the in-vitro diagnostic kit is preferably a kit for predicting and evaluating the efficacy of tumor immune drugs; and the tumor is preferably a colon cancer.

The present disclosure provides a pharmaceutical preparation, a pharmaceutical composition or a kit, comprising the fusion protein EPIL2 of the present solution.

The present disclosure provides a use of the fusion protein EPIL2 together with other drugs for treating tumors in preparation of a pharmaceutical composition or a kit for treating tumors, and the other drugs for treating tumors are preferably an anti-PD-1/PD-L1 antibody.

The present disclosure provides a use of the fusion protein EPIL2 in activating expansion of T cells.

The present disclosure provides a use of the fusion protein EPIL2 in evaluating effect/efficacy of an anti-PD-1/PD-L1 antibody drug.

The present disclosure provides a genetically engineered IL-2 having at least 3 amino acid residue substitutions; with reference to the amino acid sequence SEQ ID No.2 of the wild-type IL-2, the amino acid residue substitutions are preferably C125S, V69A, I128T, and/or Q74P; and more preferably, the amino acid sequence of the genetically engineered IL-2 is shown in SEQ ID No.6 or 8.

The present disclosure provides a separated nucleic acid molecule encoding the genetically engineered IL-2, and the nucleic acid molecule preferably has a nucleotide sequence shown in SEQ ID No.5 or 7.

The present disclosure provides an application of the genetically engineered IL-2 and/or the nucleic acid molecule encoding the genetically engineered IL-2 in the preparation of a pharmaceutical composition or an in-vitro diagnostic kit; the pharmaceutical composition is preferably a pharmaceutical composition for treating a tumor; the in-vitro diagnostic kit is preferably a kit for predicting and evaluating the efficacy of tumor immune drugs; and the tumor is preferably colon cancer.

The present disclosure also provides a tumor treatment method, comprising administering an effective amount of the fusion protein EPIL2 to a patient, or linking or sequentially administering the fusion protein and other drugs for treating tumor diseases to the patient.

### TERMS AND DEFINITIONS

Unless otherwise specified, the terms and definitions used in this application all are the meanings commonly used in the art and are known to those skilled in the art.

As used in this application, the term “tumor site” refers to an in-vivo or in-vitro location that contains or is suspected of containing tumor cells. The tumor site includes a solid tumor and a location close to or adjacent to where the tumor grows.

As used in this application, the term “administration” refers to systemic and/or local administration. The term “systemic administration” refers to non-local administration, so that the administered substance may affect a plurality of organs or tissues in the entire body; or thus the administered substance may traverse a plurality of organs or tissues in the entire body to reach the target site. For example, administration to the circulatory system of a subject may cause a therapeutic product to be expressed from the administered vector in more than one tissue or organ, or may cause the therapeutic product to be expressed at a specific site by the administered vector, for example, this is due to natural tropism or due to operable linkage with tissue-specific promoter elements. Those skilled in the art will understand that the systemic administration covers various forms of administration, including but not limited to: parenteral administration, intravenous administration, intramuscular administration, subcutaneous administration, transdermal administration, intratumoral administration, oral administration, etc.

The term “local administration” refers to administration at or around a specific site. Those skilled in the art will understand that local administration covers various forms of administration, for example, direct injection to a specific site or injection around the specific site (such as intratumoral administration).

As used herein, the term “therapeutically effective amount” refers to the interferon of the present solution required to achieve the purpose of treatment of a target disease or illness condition (for example tumor/cancer, for example for tumor regression or reduction in size of tumor), or the amount of the components in the kit of the present solution. The effective amount can be determined for a specific purpose through practices and in a conventional manner. Particularly, the therapeutically effective amount may be an amount required to achieve the following purposes: reducing the number of cancer cells; reducing the size of the tumor; inhibiting (namely slowing down or stopping) the infiltration of cancer cells into peripheral organs; inhibiting (namely slowing down or stopping) tumor metastasis; inhibiting tumor growth; and/or relieving one or more symptoms associated with cancers.

The term “antibody” covers, for example, monoclonal antibodies, polyclonal antibodies, single-chain antibodies, antibody fragments (which show the desired biological or immunological activity). In this application, the term “immunoglobulin” (Ig) and the antibody can be used interchangeably. The antibody can specifically target tumor antigens, for example, surface tumor antigens, such as EGFR, CD4, CD8, Neu and the like.

The “tumor” of the present disclosure may be selected from lung cancer, bronchial cancer, colorectal cancer, prostate cancer, breast cancer, pancreatic cancer, stomach cancer, ovarian cancer, bladder cancer, brain or central nervous system cancer, peripheral nervous system cancer, esophageal cancer, cervical cancer, melanoma, uterine or endometrial cancer, oral or throat cancer, liver cancer, kidney cancer, bile duct cancer, small intestine cancer or appendix cancer, salivary gland cancer, thymic cancer, adrenal cancer, osteosarcoma, chondrosarcoma, lipoma, testicular cancer, and malignant fibrous histiocytoma.

The “tumor cells” of the present disclosure may be selected from cells generated by lung cancer, bronchial cancer, colorectal cancer, prostate cancer, breast cancer, pancreatic cancer, stomach cancer, ovarian cancer, bladder cancer, brain or central nervous system cancer, peripheral nervous system cancer, esophagus cancer, cervical cancer, melanoma, uterine or endometrial cancer, oral or throat cancer, liver cancer, kidney cancer, bile duct cancer, small intestine cancer or appendix cancer, salivary gland cancer, thymus cancer, adrenal gland cancer, osteosarcoma, chondrosarcoma, lipoma, testicular cancer, and malignant fibrous histiocytoma cancer.

The “application” or “use” in the present disclosure may refer to applications for the purpose of disease treatment and disease treatment plan detection, and may also refer to applications for non-therapeutic purposes, such as scientific research and the like.

The beneficial effects of the present solution:
1) The fusion protein of the anti-EpCAM single-chain antibody and the IL-2 designed in the present solution can concentrate the molecular functions of interleukin-2 to pathological sites (for example, tumors) through specific targets to achieve a higher local concentration of the interleukin-2, thereby reducing the toxicity and improving the efficacy.
2) Compared with huKS-IL2, the fusion protein of the anti-EpCAM single-chain antibody and IL-2 designed in the present solution has a smaller molecular structure and shortens the distance between target molecule expressing cells and immune cells. The recognition and signal transduction of immune cells and target molecule expressing cells are promoted. Smaller molecules can achieve higher potency and have higher affinity.
3) Compared with an EpCAM/CD3-double specific antibody (for example, MT110, also known as Solitomab), the fusion proteins EPIL-2-2303301 and EPIL-2-2303407 of the present solution also provide a growth signal of the IL-2 to T cells while shortening the distance between T cells and EpCAM expressing cells (for example, tumor cells). It has been proved by tests that EPIL-2-2303301 and EPIL-2-2303407 can establish cross-linking between immune cells and EpCAM expressing cells (for example, tumor cells), and have a stronger effect of promoting proliferation of T cells than MT110.
4) Due to the better molecular penetrability, the fusion proteins EPIL-2-2303301 and EPIL-2-2303407 of the present solution can significantly improve the ability of T cells to enter tumor microspheres in a lymphocyte infiltration detection test of organoid micro-tumor models, enhance the infiltration of tumor lymphocytes, and have obviously better performances than huKS-IL2 or MT110 in promoting lymphocyte tumor infiltration.

### BRIEF DESCRIPTION OF THE DRAWINGS

FIG. 1A shows schematic diagram of the structure of protein expression vector pTT5.

FIG. 1B shows fusion protein digestion and electrophoresis identification.

FIG. 1C shows fusion protein HP-SEC purity analysis chart.

FIG. 1D shows fusion protein SDS-PAGE gel electrophoresis Coomassie Blue staining diagram.

FIG. 2 shows qualitative staining of the fusion proteins EPIL-2-2303301 and EPIL-2-2303407 bound to cellular level EpCAM.

FIG. 3 shows quantitative enzyme-linked immunoassay for the binding ability of fusion proteins EPIL-2-2303301 and EPIL-2-2303407 with EpCAM protein: the left diagram is a schematic diagram of the experimental principle, and the right diagram is a detection curve.

FIG. 4 shows stimulation of the fusion proteins EPIL-2-2303301 and EPIL-2-2303407 in proliferation of peripheral blood lymphocyte T cells.

FIG. 5 shows CSFE staining of peripheral blood lymphocyte CD8+T cell proliferation.

FIG. 6 shows stimulation of the fusion proteins EPIL-2-2303301 and EPIL-2-2303407 in proliferation of CTLL-2 cells.

FIG. 7 shows activation effect of the fusion protein EPIL-2-2303301 or EPIL-2-2303407 on phosphorylation of STAT1 and STAT3 in downstream of IL-2R.

FIG. 8 shows detection of the ability of the fusion proteins EPIL-2-2303301 and EPIL-2-2303407 to promote immune cell infiltration.

### DETAILED DESCRIPTION OF THE PREFERRED EMBODIMENTS

Hereinafter, specific embodiments of the present solution will be described in detail with reference to the drawings. It should be understood that the specific embodiments described herein are only intended to illustrate the present solution and are not intended to limit the present disclosure.

The present solution will be further described in detail through the following examples, but it should be understood that the present disclosure is not limited by the following contents.

### Example 1: Construction of Fusion Protein of Anti-EpCAM Single-Chain Antibody and IL-2

The fusion protein EPIL2 of the anti-EpCAM single-chain antibody and the IL-2, from the N end to the C end, sequentially comprises: 1) IL-2 or an IL-2 mutant, 2) a linker, and 3) an anti-EpCAM single-chain antibody. The amino acid sequence of the IL-2 or the IL-2 mutant is shown in SEQ ID No.2, 4, 6 and 8; the amino acid sequence of the linker is shown in SEQ ID No.10; and the amino acid sequence of the anti-EpCAM single-chain antibody is shown in SEQ ID No.11 and 13.

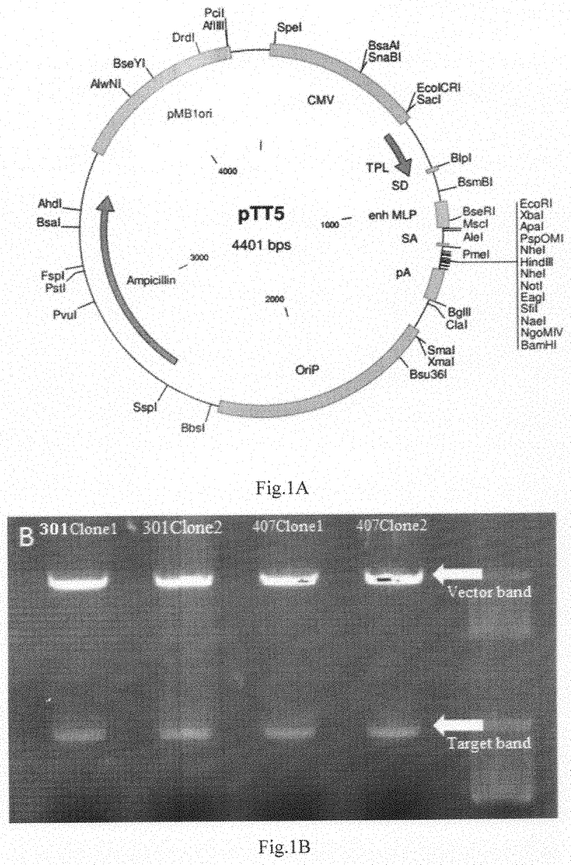

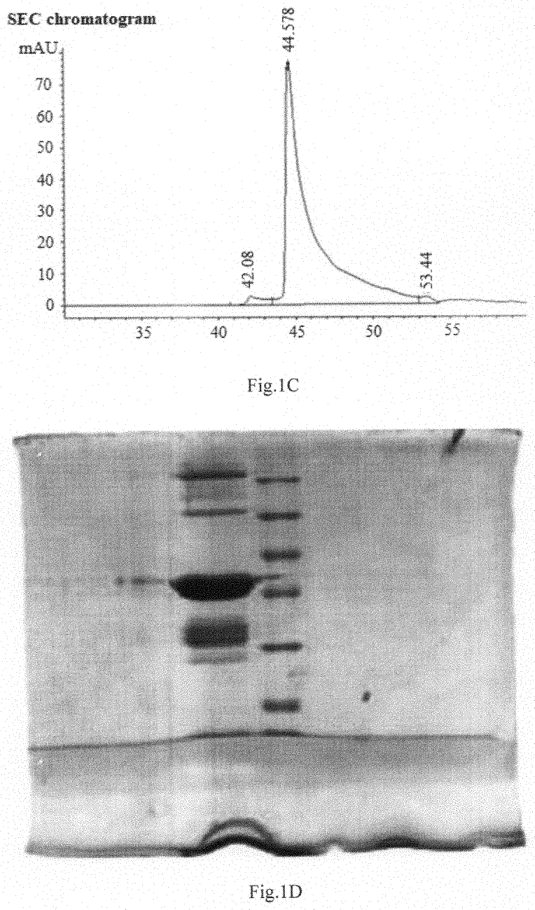

The DNA sequence of the fusion protein of the anti-EpCAM single-chain antibody and the IL-2 is synthesized by adopting a complete-sequence gene synthesis method to obtain a target DNA sequence (SEQ ID No.16 and 18), and after synthesis, the sequence is connected to a T vector, and a pTT5 plasmid vector and a pEASY-T1 positive clone that is equipped with a target sequence are subjected to double digestion using HindIII and NheI. A long fragment of the vector and a target fragment cut from the T vector are recycled and are linked to form a target clone using ligase, and the positive clone (FIG. 1B) is identified by double digestion using HindIII and NheI.

The specific steps are as follows: after the design of the target gene fragment is completed, synthesis of the target gene fragment is implemented by a commercial gene synthesis company, and PCR identification is implemented through a T vector (pEASY-T1 Cloning Vector, TransGen, Product Number CT101) by using an M13 forward primer and an M13 reverse primer. The positive clone is further sequenced through the M13 forward primer.

2 ug of a cloned plasmid and 2 ug of a pTT5 plasmid identified by sequencing are prepared; 5 ul of buffer 1.1 (Brand: NEB, Product Number #B7201S) is added; 2 ul of HindIII (Brand: NEB, Product Number #R0104) restriction endonuclease and 2 ul of NheI (Brand: NEB, Product Number #R0131) restriction endonuclease are correspondingly added; and double-distilled water is added until the volume is 50 ul. Incubation is performed in a water bath at 37° C. for 2 hours; 5 ul of electrophoresis loading buffer is added; 1.2% agarose gel is added; electrophoresis is performed to separate the digested fragments; and the large fragment of pTT5 and the target vector fragment in the T vector are recycled.

Mixing is performed according to a molar ratio of 1:3, afterwards ligase and ligase buffer are added; the mixture is kept at room temperature for 5 minutes and is then inserted into ice for later use. Competent cells (Trans 1-T1) are taken out from an ultra-low temperature (−70° C.) refrigerator and unfrozen on the ice surface; a ligation product is added; mixing is performed gently; the mixture is put on ice for 30 minutes and is thermally activated at 42° C. for 30 seconds and is then inserted on ice to be cooled for 2 minutes; afterwards 250 ul of a room-temperature LB medium is added; and culturing is performed at 200 rpm at 37° C. for 1 hour.

200 ul of a bacterial solution is evenly smeared on a prepared panel. The panel is inverted in a 37° C. incubator for culturing for 12-16 hours; clones are selected and put into 10 microliters of sterile water; mixing is performed in a whirling manner; 1 microliter of the mixed liquor is put into 25 microliters of a PCR system; and the positive clone is identified by using the M13 forward primer and the M13 reverse primer. The PCR procedure is: operation is performed at 94° C. for 10 minutes, cycled 30 times (at 94° C. for 30 seconds, at 55° C. for 30 seconds, at 72° C. for 60 seconds), and performed at 72° C. for 10 minutes.

The positive clone is inoculated into an LB medium; after culturing is performed at 37° C. and at 120 rpm for 16 hours, the plasmid is extracted; 0.5 ug of the plasmid is subjected to double digestion using HindIII and NheI and is then subjected to agarose gel electrophoresis identification (FIG. 1B).

High-purity plasmids adopt a low-endotoxin plasmid large-scale extraction kit (Brand: Qiagen, Product Number: #12362). Purification methods follow the steps provided by the manufacturer. The brief process is as follows: the expression strain of the positive clone is cultured in an LP liquid medium at 37° C. and at a rotation speed of 200 rpm; and after overnight culturing, the expression strain is centrifuged at 6000 g for 15 minutes to obtain bacterial strain precipitate. Lysis buffer is added according to the instruction of the kit and is neutralized, and the supernatant is collected after centrifugation. The supernatant is added to the column; after washing buffer is added, elution buffer is added to elute the DNA of the plasmid, isopropanol is used for precipitation; the precipitate is collected after centrifugation is performed at 15000 g for 30 minutes; the precipitate is dried in air after being washed using ethanol; pure water is added to dissolve the DNA; and after quantification, the DNA is saved in a frozen state or used to transfect cells.

### Example 2: Expression and Purification of Fusion Protein of Anti-EpCAM Single-Chain Antibody and IL-2

The low-endotoxin large-scale extraction plasmid is used to transfect HEK293 cells by a liposome method, and the specific steps are: inoculating 6×10⁶ HEK293 cells in 20 ml of a DMEM (+10% FBS+antibiotic) medium; culturing the cells at 37° C., at 175 rpm, and under a condition of 5% CO₂ for 3 days; getting the cell count, when the cell density is about 2×10⁶ to 3×10⁶ cells/ml, carrying out a dilution treatment with a fresh DMEM (+10% FBS, no antibiotics) medium until the cell density reaches 1×10⁶ cells/ml, wherein the volume of each bottle of cell fluid is 20 mL; then screwing the opening of the bottle and placing it in a shaker to continue the culturing, and performing transfection after 2-4 hours; in a sterile test tube, diluting 20 ug of plasmids using 500 ul of DMEM; in another test tube, 50 ul of lipofectamine 2000 (Brand: invitrogen, Product Number: #11668-027) is added; after standing at room temperature for 5 minutes, uniformly mixing the plasmids with the liposomes, and afterwards standing at room temperature for 20 minutes; adding the mixture to a cell culture flask and continuing to culture for 4 hours; collecting cells by centrifugation, and adding 20 ml of a fresh DMEM (+10% FBS, +antibiotics) medium; continuing to culture, amplifying the system to 5 L, then continuing to culture for 5-7 days, and checking the cell density and cell viability at any time during culturing; obtaining 5 L of HEK293 cell culture liquid, and centrifuging at 10000 rpm and at 4° C. for 10 minutes; collecting the supernatant, and filtering it using a 0.22 um capsule filter.

Equilibrating a chromatography column on an AKTA Purifier 100 purification system, using an XK26/20 chromatography column of which the volume is 30 ml, and using a GE Healthcare Ni-Sepharose chromatography medium; adopting equilibration buffer which is 50 mM NaH₂PO₄₋ 300 mM NaCl of which the pH is equal to 7.4, and performing equilibration for 10 CV; loading and purifying the cell supernatant collected after filtration on the AKTA Purifier 100 purification system, wherein the sample loading flow rate is 20 ml/min; after loading of the sample is completed, using the equilibration buffer to perform washing and equilibration again, and washing the chromatography column for 10CV; using the AKTA Buffer configuration function, and using 50 mM NaH₂PO₄— 300 mM NaCl −5 mM imidazole of which the pH is equal to 7.4 for washing to remove impurity proteins and DNA pollutants; performing elution by respectively using buffer with different imidazole concentrations, such as 50 mM NaH₂PO₄₋ 300 mM NaCl −50 mM imidazole of which the pH is equal to 7.4, 50 mM NaH₂PO₄₋ 300 mM NaCl −100 mM imidazole of which the pH is equal to 7.4, 50 mM NaH₂PO₄₋ 300 mM NaCl −200 mM imidazole of which the pH is equal to 7.4, 50 mM NaH₂PO_{4—} 300 mM NaCl −500 mM imidazole of which the pH is equal to 7.4 and the like, and collecting the protein purification peak obtained by corresponding elution; collecting the purified proteins, respectively sampling the proteins for subsequent SDS-PAGE analysis and SEC-HPLC purity analysis; and using an ultrafiltration system to replace the target protein buffer with PBS buffer of which the pH is equal to 7.4; after purification, performing the regeneration of the chromatography column according to the following procedure: a. adding deionized water with a volume which is 5 times the column volume to wash the column once; b. adding 2% SDS with a volume which is 3 times the column volume to wash the column once; c. adding 25%, 50%, and 75% ethanol with a volume which is 1 time the column volume to wash the column once, respectively; d. adding 100% ethanol with a volume which is 5 times the column volume to wash the column once; e. adding 75%, 50%, and 25% ethanol with a volume which is 1 time the column volume to wash the column once, respectively; f. adding deionized water with a volume which is 2 times the column volume to wash the column once; g. subsequently storing the His-tag Purification chromatography column in 20% ethanol at 4° C.;

The fusion proteins EPIL2-2303301 (SEQ ID No.15) and EPIL2-2303407 (SEQ ID No.17) of the anti-EpCAM single-chain antibody and the IL-2 are obtained.

### Example 3: Affinity Detection of Fusion Protein of Anti-EpCAM Single-Chain Antibody and IL-2

1. Qualitative verification of the binding ability of the fusion proteins EPIL-2-2303301 and EPIL-2-2303407 with cellular level EpCAM:

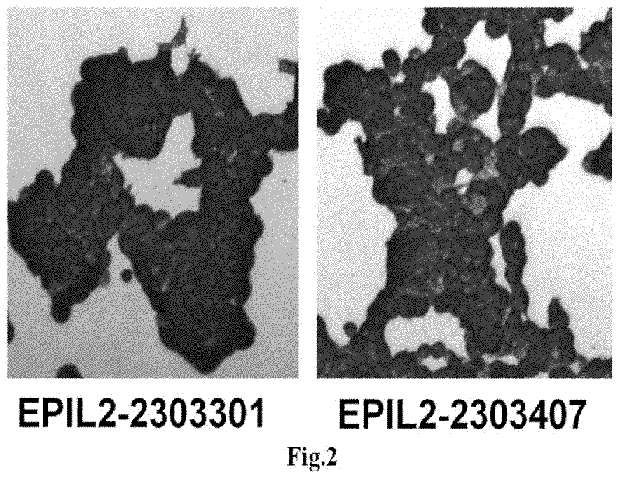

Skov3 cells are stained to test the affinity of EPIL-2-2303301 and EPIL-2-2303407 to the EpCAM protein thereon. Skov3 cells have adherent growth in a 24-pore plate for 24 hours and are fixed for 10 minutes using 4% PFA (paraformaldehyde); 5% BSA is added to block non-specific antigens; a primary antibody of anti-human IL-2 is added for incubation for 1 hour; after washing is performed 3 times with PBST, a secondary antibody is added; after incubation is performed for 1 hour, DAB (diaminobenzidine) is added for color development; and after washing is performed with PBS, photographing is performed under a bright-field microscope. It can be seen that EPIL-2-2303301 and EPIL-2-2303407 can form quite strong cell staining, qualitatively proving their binding ability with the EpCAM at the cellular level (FIG. 2).

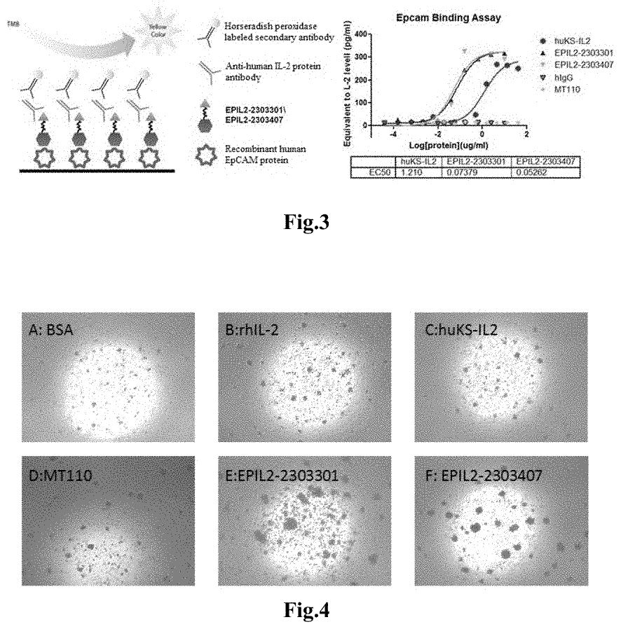

2. Quantitative enzyme-linked immunoassay for the binding ability of the fusion proteins EPIL-2-2303301 and EPIL-2-2303407 with EpCAM protein:
i. Enzyme-labeled plate coating: diluting a recombinant human EpCAM protein (Manufacturer: Sino biologics; Product Number 10694-H08H) to 1 ug/ml using PBS (phosphate buffered saline); adding 100 ul to a 96-pore high-adsorption enzyme-labeled plate (Manufacturer: Corning; Product Number #3590); placing at 37° C. for 1 hour; washing 3 times using PBS buffer; adding 100 ul of PBS buffer containing 1% BSA to perform sealing for 1 hour; adding gradually diluted huKS-IL2, MT110, EPIL-2-2303301, EPIL-2-2303407 and human IgG (negative control); placing it at 25° C. for 1 hour; and adding 200 ul of PBST buffer (phosphate buffered saline with 0.05% Tween-20) to wash 3 times.
ii. IL-2 level detection: an IL-2 ELISA commercial kit (Manufacturer: Biolegend; Product Number 431801) is adopted; the brief steps include adding a primary antibody of anti-human IL-2, incubating for 1 hour and then adding a horseradish peroxidase labeled secondary antibody to obtain a chemiluminescent signal by TMB color development.
iii. Data processing: a standard curve is obtained by using an IL-2 standard product included in the kit, and the IL-2 signal level corresponding to the amount of protein bound to EpCAM is calculated, which represents the level of the protein bound to EpCAM. The data shows that EPIL-2-2303301 and EPIL-2-2303407 have a small protein molecular weight and a high level of affinity. MT110 itself does not have IL-2, so it cannot be identified in this detection (FIG. 3).

### Example 4: Verification of Biological Activity of Fusion Protein of Anti-EpCAM Single-Chain Antibody and IL-2

1. Separation of Peripheral Blood Lymphocytes:

Getting peripheral blood provided by healthy donors; drawing 20 ml of venous blood of healthy donors into a heparin anticoagulation blood collection tube; adding 20 ml of phosphate buffered saline (PBS) to 20 ml of freshly collected peripheral blood; after gently mixing, adding 9 ml to a 15 ml centrifuge tube containing 5 ml of lymphocyte separation solution (GE Ficoll-Paque PLUS); carefully gently adding it along the wall of the centrifuge tube to avoid shaking to form a lower layer lymphocyte separation solution and realize two-image layering of upper layer blood cells, centrifuging at 2000 rpm for 30 minutes, and taking the lymphocyte layer; adding 2 times the volume of RPMI1640 medium (2 mM L-glutamine, 5% FBS), centrifuging at 400 g for 5 minutes, discarding the supernatant, subsequently repeating washing twice, afterwards using the RPMI1640 medium (added with 10% FBS, 2 mM L-glutamine, 1×double antibody), performing cell counting, adjusting the concentration to 1×10⁶ cells/ml, and obtaining a total of 18.6×10⁶ peripheral blood lymphocytes. Peripheral blood lymphocytes can be cryopreserved or immediately applied to follow-up experiments.

2. Peripheral Blood Lymphocyte T Cell Proliferation Experiment

In a 6-pore plate, adding 1 ml of 1 ug/ml anti-CD3 monoclonal antibody (clone: OKT3, Manufacturer: eBioscience, Product Number: 14-0037-82) to each pore, shaking gently, covering the bottom, placing in a 37° C. incubator and incubating for 1 hour, afterwards adding 2 ml PBS, and washing twice for later use; resuspending freshly separated PBMC or cryopreserved PBMC in a lymphocyte culture medium (RPMI1640 medium with 10% FBS, 2 mM L-glutamine, 1×double antibody), treating the preheated 6-pore cell culture plate before adding, and incubating at 37° C. for 2 hours; collecting the suspended cells, collecting the cells by centrifugation, resuspending the cells in the lymphocyte culture medium, adjusting the cell density to 2×10⁵ cells/ml, in a 96-pore cell culture plate, and adding BSA, rhIL-2 (Recombinant Human IL-2, Manufacturer: Peprotech, Product Number: 200-02), huKS-IL2 (Manufacturer: Creative Biolabs, Product Number ACFP-SH034), MT110 (Manufacturer: Creative Biolabs, Product Number TAB-889), EPIL-2-2303301 or EPIL-2-2303407 with a final concentration of 1 ng/ml; placing the cell culture plate in a cell incubator which is at 37° C. and contains 5% carbon dioxide; culturing for 4-7 days; and taking photos to observe the quantity and size of clone clusters generated by T cell expansion in PBMC.

This experiment aims to verify whether the IL-2-anti-EpCAM single-chain antibody fusion proteins EPIL-2-2303301 and EPIL-2-2303407 have the cell biological activity of IL-2. The abilities of recombinant human IL-2, huKS-IL2, MT110, EPIL-2-2303301 and EPIL-2-2303407 to promote T cell proliferation are compared.

As shown in FIG. 4, EPIL-2-2303301 and EPIL-2-2303407 can promote the expansion of peripheral blood lymphocytes. The ability to promote the expansion of peripheral blood lymphocytes is more active than that of the recombinant human interleukin-2, huKS-IL2 or MT110. It can be seen from the size and quantity of the clones generated by T cell expansion that in the peripheral blood lymphocytes treated with EPIL-2-2303301 or EPIL-2-2303407, the clone clusters generated by T cell expansion are more and larger, representing that the T cell expansion speed is higher. In the control group, as the clone clusters of T cells are quite small, the expansion is not obvious. Comparative recombinant human interleukin-2 (rhIL2), huKS-IL2 or MT110-treated peripheral blood lymphocytes have clone clusters generated by T cell expansion, but the clone clusters are more and smaller in size than that in EPIL-2-2303301 or EPIL-2-2303407 treated peripheral blood lymphocytes. The test data shows that EPIL-2-2303301 or EPIL-2-2303407 has a quite strong ability to promote the expansion of T lymphocytes.

3. CF SE staining Experiment of Peripheral Blood Lymphocyte CD8+T Cell Proliferation

As expansion is realized with mitosis of T cells, the color of CF SE staining is gradually dispersed, and the degree of CSFE signal reduction reflects the expansion activity of T cells. As described in the previous step, the separated human peripheral blood lymphocytes (PBMC) are washed 3 times with PBS containing 2% FBS (fetal bovine serum). The cells are resuspended with PBS containing 2.5 uM CSFE, and are incubated for 10 minutes at 37° C. 2% FBS is added to stop staining, and washing is performed twice with the 2% FBS. The cells are resuspended in the lymphocyte culture medium (RPMI1640 medium added with 10% FBS, 2 mM L-glutamine, 1× double antibody). The cells are added to a cell culture plate coated with an OKT3 antibody, and BSA, rhIL-2 (recombinant human IL-2, Manufacturer: Peprotech, Product Number: 200-02), HuKS-IL2 (Manufacturer: Creative Biolabs, Product Number ACFP-SH034), MT110 (Manufacturer: Creative Biolabs, Product Number TAB-889), EPIL-2-2303301 or EPIL-2-2303407 with a final concentration of 1 ng/ml are added to the corresponding cell pores. The cell culture plate is placed in a cell incubator which is at 37° C. and contains 5% carbon dioxide. Culturing is performed for 6 days. After 6 days of culturing, the cells are collected and fixed, afterwards CD8+T cells are marked with an anti-CD8-APC labeled fluorescent antibody. FACS uses a GRN channel to detect a green signal of CF SE. The dilution degree of CF SE in CD8+T cells is analyzed, which represents the proliferation condition of CD8+T cells.

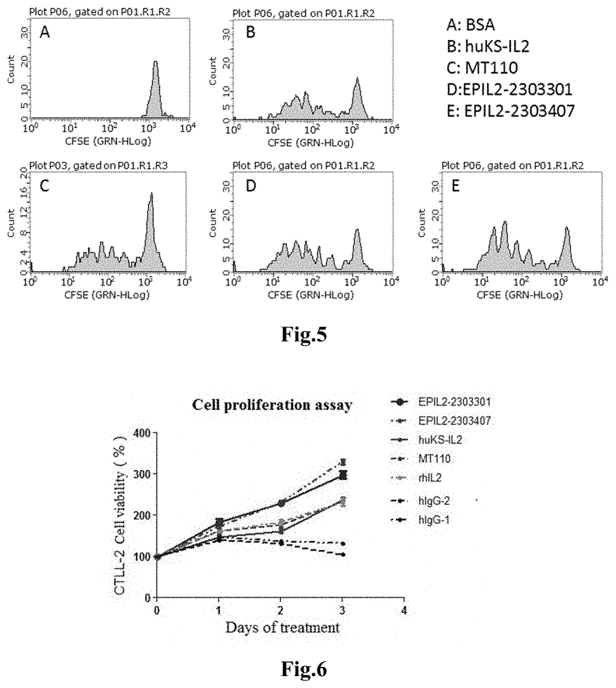

As shown in FIG. 5, EPIL-2-2303301 or EPIL-2-2303407 promotes the expansion of CD8+T cells, and in the CFSE staining test, EPIL-2-2303301 or EPIL-2-2303407 has higher activity than the recombinant human interleukin-2, huKS-IL2 or MT110.

4. CTLL-2 Cell Proliferation Assay

CTLL-2 (ATCC Product Number TIB-214) is cultured in the RPMI1640 lymphocyte culture medium (RPMI1640 added with 10% FBS, 2 mM L-glutamine, 1×double antibody); when the cells are in a high density and growing vigorously, a 1640 culture solution is used to wash the cells 3 times, and the RPMI1640 lymphocyte culture medium is used to prepare 2×10⁵/ml cell suspension. 50 ul of cell suspension is added into the 96-pore cell culture plate. The corresponding cell pores are added with 50 ul of IgG, rhIL-2 (Recombinant Human IL-2, Manufacturer: Peprotech, Product Number: 200-02), huKS-IL2 (Manufacturer: Creative Biolabs, Product Number ACFP-SH034), MT110 (Manufacturer: Creative Biolabs, Product Number TAB-889), EPIL-2-2303301 or EPIL-2-2303407 that all have a concentration of 100 ng/ml. The cell culture plate is placed in a cell incubator which is at 37° C. and contains 5% carbon dioxide. Culturing is performed for 3 days. Cell viability is measured once a day. The 96-pore plate is taken out; the intraday pores are added with 50 ul CellTiter-Glo® (Promega, Cat. No. G7571) cell lysis liquid. After shaking and mixing is performed for 5-10 minutes, the level of chemiluminescence is measured in a plate reader (BMG Omega) with a chemiluminescence program (luminescence). The horizontal axis of the cell proliferation curve represents time (days), and the vertical axis represents the corresponding cell viability.

As shown in FIG. 6, EPIL-2-2303301 and EPIL-2-2303407 promote the expansion of CTLL-2 cells, and in the CTLL-2 cell proliferation test, EPIL-2-2303301 or EPIL-2-2303407 has higher activity than the recombinant human interleukin-2, HuKS-IL2 or MT110.

### Example 5: In-Vivo and In-Vitro Application Schemes of Fusion Protein of Anti-EpCAM Single-Chain Antibody and IL-2

1.IL-2/STAT1/3 Signal Channel Experiment

IL-2 is bound to IL-2Rα (CD25) and IL-2β on the cell surface to activate the phosphorylation of STAT protein. In the present solution, CTLL-2 cells are used to verify the activation effect of huKS-IL2, EPIL-2-2303301 or EPIL-2-2303407 on the phosphorylation of STAT1 and STAT3 in downstream of IL-2R.

CTLL-2 (ATCC Product Number TIB-214) is cultured in the RPMI1640 lymphocyte culture medium (RPMI1640 added with 10% FBS, 2 mM L-glutamine, 1×double antibody); when the cells are in a high density and growing vigorously, the CTLL-2 cells are collected by centrifugation and centrifuged at 400 g for 3 minutes; the supernatant is discarded; PBS is added; washing and counting are performed; 1×10⁷ cells are transferred to a new centrifuge tube to be centrifuged at 400 g for 3 minutes; cell pellets are collected; a low-serum RPMI1640 lymphocyte medium (RPMI1640 added with 0.5% FBS, 2 mM L-glutamine, 1×double antibody) is used to resuspend the cells; the cells are transferred to a cell culture flask; the cell culture flask is placed in an incubator which is at 37° C. and contains 5% carbon dioxide to perform incubation for 4 hours; afterwards the cells are collected again; and 4.2 ml of serum-free RPMI1640 lymphocyte medium (RPMI1640, 2 mM L-glutamine, 1×double antibody) is used to resuspend the cells. 1 ml of cell suspension is transferred to a new centrifuge tube and is treated by respectively adding 1 ng/ml bovine serum albumin (BSA, low endotoxin, Manufacturer: Sigma, Product Number A1933), MT110 (Manufacturer: Creative Biolabs, Product Number TAB-889), 1 ng/ml recombinant human interleukin-2 (Recombinant Human IL-2, Manufacturer: Peprotech, Product Number: 200-02), 1 ng/ml huKS-IL2, 1 ng/ml EPIL-2-2303301 or 1 ng/ml EPIL-2 -2303407 at 37° C. for 20 minutes. The cells are collected by centrifugation; 100 ul of RIPA lysis solution (50 mM Tris-HCl (pH 7.4), 150 mM NaCl, 1% NP-40, 0.1% SDS) is added; centrifugation is performed at 12000 g at 4° C. for 10 minutes; and the supernatant is transferred to a new centrifuge tube to obtain the total protein product of cell lysis. After measuring the concentration of the protein, the protein is separated by 10% SDS polyacrylamide gel electrophoresis. After the protein is transferred to a PVDF membrane, the phosphorylated STAT3, phosphorylated STAT1 and GAPDH are tested using an antibody. Phosphorylated STAT3 antibody (Manufacturer: CST, Product Number: #9145), phosphorylated STAT3 antibody (Manufacturer: CST, Product Number: #9351), and GAPDH antibody (Manufacturer: CST, Product Number: #5174).

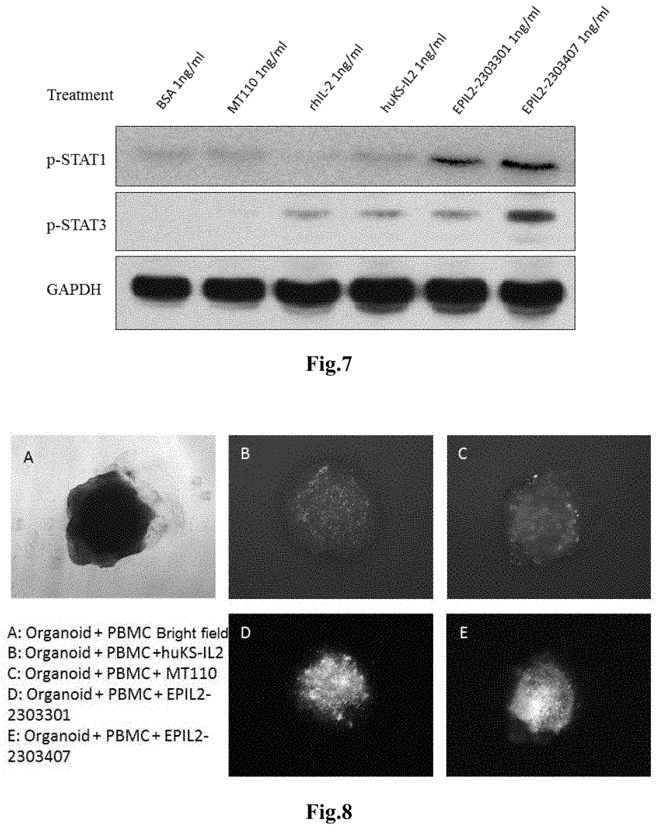

As shown in FIG. 7, EPIL-2-2303301 or EPIL-2-2303407 activates a signaling channel in downstream of IL2R. EPIL-2-2303301 or 1 ng/ml EPIL-2-2303407 activates the phosphorylation of STAT1 and STAT3; when EPIL-2-2303301 or 1 ng/ml EPIL-2-2303407 is compared with the recombinant human interleukin-2, huKS-IL2 or MT110, MT110 and the control group have no activity, the recombinant human interleukin-2 and huKS-IL2 have certain activity, but the activity is lower than that of EPIL-2-2303301 or EPIL-2-2303407.

2. Detection of Lymphocyte Infiltration Ability

Tumor tissues are an integral whole in the body, which consist of tumor epithelial cells, tumor-related fibrocytes, immune cells and the like. Tumors may generate immunogenicity and induce the immune system to recognize and attack tumor cells, which is the principle of tumor immunotherapy. However, a tissue structure with concentrated tumors often prevents immune cells from entering its core area, or forms a micro-environment that suppresses the immune system. Therefore, enhancing the entry of immune cells into tumors is a key factor for improving the effectiveness of tumor immunotherapy drugs.

The present solution uses a tumor organoid model (organoids) to simulate the tissue structure in the tumor, and the organoid model is a 3D stem cell culture technology that can simulate the tissue structure of the tumor. In the present disclosure, a colon cancer organoid is used to simulate in-vivo tumors to evaluate the effect of a bioactive agent on immune cell infiltration. The “bioactive agent” herein refers to huKS-IL2, MT110, EPIL-2-2303301 or EPIL-2-2303407.

The specific scheme is as follows: the colon cancer tumor organoid is cultured from surgically resected colon cancer tumor tissues. A fresh tumor sample is cut into a minced shape and digested at 37° C. for 30 minutes by using collagenase 4 (75 U/mL) and neutral protease 2 (125 ug/ml). Large pieces of tissues are removed, and the supernatant is centrifuged at 200 g for 3 minutes at 4° C. . The cell pellets are collected, added with matrigel with reduced growth factors, dripped into a 48-pore cell culture plate (25 μl/pore) and incubated at 37° C. for 37 minutes, and then the upper layer is added to a colon cancer organoid culture medium (Advanced DMEM/F12 medium added with 10 mM HEPES buffer, 2 mM glutamine, B27 cell culture additive, 10 nM gastrin, 1 mM N-acetylcysteine, 50 ng/ml epidermal growth factor, 100 ng/ml Noggin, 10% R-spondin-1 conditioned medium, and 50% Wnt-3A conditioned medium). In a 37° C. cell incubator containing 5% carbon dioxide, culture solution is supplemented once every 2 days; and after the organoids grow for 7-20 days, the tumor organoids are carefully sucked out of a culture dish with a glass pipette and placed in a 96-pore U-shaped low-adsorption culture plate of a lymphocyte basic culture medium (RPMR-1640+10% fetal bovine serum+penicillin streptomycin double antibody). Freshly separated human peripheral blood lymphocytes (PBMC) are stained using CFSE (method, see above), and the CFSE stained lymphocytes are added according to 1×10⁵ cells/pore. The pores are added with huKS-IL2, MT110, EPIL-2-2303301 or EPIL-2-2303407 with a final concentration of 1 ng/ml, and are placed at 37° C. for 4 hours. After 4 hours, the colon cancer organoids are carefully taken out of the pores and placed in a new 24-pore flat-bottomed culture dish. A bright field and a green fluorescence microscope are used to observe green signals. Green cells in the organoids represent the quantity of immune cells that infiltrate into the tumor.

As shown in FIG. 8, EPIL-2-2303301 and EPIL-2-2303407 have a higher ability to promote immune cell infiltration than huKS-IL2 or MT110.

The above examples are only preferred examples of the present invention and are not intended to limit the present invention. Any modification, equivalent replacement, improvement, etc. made within the spirit and principle of the present invention should be included within the protection scope of the present solution.

The preferred embodiments of the present invention have been described in detail with reference to the accompanying drawings. However, the present invention is not limited to the specific details in the above embodiments. Within the scope of the technical concept of the present invention, the technical schemes of the present invention can be subjected to simple modifications, and these simple modifications all belong to the protection scope of the present invention.

Further to be noted that, in various specific features of the above-described specific embodiments described, may be combined in any suitable manner without conflict. To avoid unnecessary repetition, the present invention will not further descript the various possible combinations.

Further, among various embodiments of the present invention may be arbitrarily combined as long as it does not violate the spirit of the invention, which should also be considered as the disclosure of the present invention.

What is claimed is:

1. A fusion protein, characterized in that the fusion protein is composed by interleukin-2 (IL-2) being fused with and linked to an anti-human epithelial cell adhesion molecule (EpCAM) single-chain antibody.

2. The fusion protein according to claim 1, characterized in that the interleukin-2 (IL-2) is a wild-type IL-2 or a genetically engineered IL-2.

3. The fusion protein according to claim 2, characterized in that the genetically engineered IL-2 has at least 3 amino acid residue substitutions; with reference to the amino acid sequence of the wild-type IL-2, the amino acid residue substitutions are selected from C125S, V69A, I128T, and/or Q74P.

4. The fusion protein according to claim 2, characterized in that the amino acid sequence of the wild-type IL-2 is shown in SEQ ID No.2;
the amino acid sequence of the genetically engineered IL-2 is shown in SEQ ID No.4, 6 or 8.

5. The fusion protein according to claim 1, characterized in that the amino acid sequence of the anti-human epithelial cell adhesion molecule (EpCAM) single-chain antibody is shown in SEQ ID No.11 or 13.

6. The fusion protein according to claims 1, characterized in that the amino acid sequence of the fusion protein is shown in SEQ ID No.15 or 17.

7. A separated nucleic acid molecule encoding the fusion protein composed by interleukin-2 (IL-2) being fused with and linked to an anti-human epithelial cell adhesion molecule (EpCAM) single-chain antibody, characterized in that the nucleic acid molecule comprises a nucleotide sequence shown in SEQ ID No.16 or 18.

8. A genetically engineered IL-2, characterized in that the genetically engineered IL-2 has at least 3 amino acid residue substitutions; with reference to the amino acid sequence SEQ ID No.2 of the wild-type IL-2, the amino acid residue substitutions are selected from C125S, V69A, I128T, and/or Q74P.

9. The genetically engineered IL-2 according to claim 8, characterized in that the amino acid sequence of the genetically engineered IL-2 is shown in SEQ ID No.6 or 8.


SEQUENCE LISTING (19 sequences)

SEQ ID NO: 1 - DNA, 405 bp, Homo sapiens
atggcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 2 - protein, 134 aa, Homo sapiens
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 3 - DNA, 462 bp, Homo sapiens
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 4 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 5 - DNA, 462 bp, Artificial Sequence
  coding IL-2 mutant I containing T433A (C125S), T266C (V69A), T443C (I128T)
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagcgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcaccatctcaacactgacttga

SEQ ID NO: 6 - protein, 153 aa, Artificial Sequence
  IL-2 muntant I containing T433A (C125S), T266C (V69A), T443C (I128T)
MYRMQLLSCIALSLALVTNSAPTSSSTKKTQLQLEHLLLDLQMILNGINNYKNPKLTRMLTFKFYMPKKATELKHLQCLEEELKPLEEALNLAQSKNFHLRPRDLISNINVIVLELKGSETTFMCEYADETATIVEFLNRWITFSQSTISTLT

SEQ ID NO: 7 - DNA, 462 bp, Artificial Sequence
  coding IL-2 mutant II containing T433A (C125S), T266C (V69A), A281C (Q74P), T443C (I128T)
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagcgctaaatttagctccaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcaccatctcaacactgacttga

SEQ ID NO: 8 - protein, 153 aa, Artificial Sequence
  IL-2 mutant II containing T433A (C125S), T266C (V69A), A281C (Q74P), T443C(I128T)
MYRMQLLSCIALSLALVTNSAPTSSSTKKTQLQLEHLLLDLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSTISTLT

SEQ ID NO: 9 - DNA, 60 bp, Artificial Sequence
  (G4S)4 linker
ggtggtggtggttctggcggcggcggctccggtggtggtggttctggcggcggcggctcc

SEQ ID NO: 10 - protein, 20 aa, Artificial Sequence
  (G4S)4 linker
GGGGSGGGGSGGGGSGGGGS

SEQ ID NO: 11 - protein, 248 aa, Artificial Sequence
  3-1 VL-VH
EVQLLEQSGAELVKPGASVKISCKASGYAFTNYWLGWVKQRPGHGLEWIGDLFPGSGNTHYNERFRGKATLTADKSSSTAFMQLSSLTSEDSAVYFCARLRNWDEAMDYWGQGTTVTVSSGGGGSGGGGSGGGGSELVMTQSPSYLAASPGETITINCRASKSISKYLAWYQEKPGKTNKLLIYSGSTLQSGIPSRFSGSGSGTDFTLTISSLEPEDFAMYYCQQHNEYPYTFGGGTKLEIKHHHHHH

SEQ ID NO: 12 - DNA, 747 bp, Artificial Sequence
  3-1 VL-VH
gaggtgcagctgctcgagcagtctggagctgagctggtgaaacctggggcctcagtgaagatatcctgcaaggcttctggatacgccttcactaactactggctaggttgggtaaagcagaggcctggacatggacttgagtggattggagatcttttccctggaagtggtaatactcactacaatgagaggttcaggggcaaagccacactgactgcagacaaatcctcgagcacagcctttatgcagctcagtagcctgacatctgaggactctgctgtctatttctgtgcaagattgaggaactgggacgaggctatggactactggggccaagggaccacggtcaccgtctcctcaggtggtggtggttctggcggcggcggctccggtggtggtggttctgagctcgtcatgacccagtctccatcttatcttgctgcatctcctggagaaaccattactattaattgcagggcaagtaagagcattagcaaatatttagcctggtatcaagagaaacctgggaaaactaataagcttcttatctactctggatccactttgcaatctggaattccatcaaggttcagtggcagtggatctggtacagatttcactctcaccatcagtagcctggagcctgaagattttgcaatgtattactgtcaacagcataatgaatatccgtacacgttcggaggggggaccaagcttgagatcaaacatcatcaccatcatcattag

SEQ ID NO: 13 - protein, 257 aa, Artificial Sequence
  4-7 VL-VH
EVQLLEQSGAELARPGASVKLSCKASGYTFTNYGLSWVKQRPGQVLEWIGEVYPRIGNAYYNEKFKGKATLTADKSSSTASMELRSLTSEDSAVYFCARRGSYDTNYDWYFDVWGQGTTVTVSSGGGGSGGGGSGGGGSELVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKHHHHHH

SEQ ID NO: 14 - DNA, 774 bp, Artificial Sequence
  4-7 VL-VH
gaggtgcagctgctcgagcagtctggagctgagctggcgaggcctggggcttcagtgaagctgtcctgcaaggcttctggctacaccttcacaaactatggtttaagctgggtgaagcagaggcctggacaggtccttgagtggattggagaggtttatcctagaattggtaatgcttactacaatgagaagttcaagggcaaggccacactgactgcagacaaatcctccagcacagcgtccatggagctccgcagcctgacctctgaggactctgcggtctatttctgtgcaagacggggatcctacgatactaactacgactggtacttcgatgtctggggccaagggaccacggtcaccgtctcctcaggtggtggtggttctggcggcggcggctccggtggtggtggttctgagctcgtgatgacccagactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagcttgagatcaaacatcatcaccatcatcattag

SEQ ID NO: 15 - protein, 421 aa, Artificial Sequence
  EPIL-2-2303301
MYRMQLLSCIALSLALVTNSAPTSSSTKKTQLQLEHLLLDLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSTISTLTGGGGSGGGGSGGGGSGGGGSEVQLLEQSGAELVKPGASVKISCKASGYAFTNYWLGWVKQRPGHGLEWIGDLFPGSGNTHYNERFRGKATLTADKSSSTAFMQLSSLTSEDSAVYFCARLRNWDEAMDYWGQGTTVTVSSGGGGSGGGGSGGGGSELVMTQSPSYLAASPGETITINCRASKSISKYLAWYQEKPGKTNKLLIYSGSTLQSGIPSRFSGSGSGTDFTLTISSLEPEDFAMYYCQQHNEYPYTFGGGTKLEIKHHHHHH

SEQ ID NO: 16 - DNA, 1266 bp, Artificial Sequence
  EPIL-2-2303301
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagcgctaaatttagctccaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcaccatctcaacactgactggtggtggtggttctggcggcggcggctccggtggtggtggttctggcggcggcggctccgaggtgcagctgctcgagcagtctggagctgagctggtgaaacctggggcctcagtgaagatatcctgcaaggcttctggatacgccttcactaactactggctaggttgggtaaagcagaggcctggacatggacttgagtggattggagatcttttccctggaagtggtaatactcactacaatgagaggttcaggggcaaagccacactgactgcagacaaatcctcgagcacagcctttatgcagctcagtagcctgacatctgaggactctgctgtctatttctgtgcaagattgaggaactgggacgaggctatggactactggggccaagggaccacggtcaccgtctcctcaggtggtggtggttctggcggcggcggctccggtggtggtggttctgagctcgtcatgacccagtctccatcttatcttgctgcatctcctggagaaaccattactattaattgcagggcaagtaagagcattagcaaatatttagcctggtatcaagagaaacctgggaaaactaataagcttcttatctactctggatccactttgcaatctggaattccatcaaggttcagtggcagtggatctggtacagatttcactctcaccatcagtagcctggagcctgaagattttgcaatgtattactgtcaacagcataatgaatatccgtacacgttcggaggggggaccaagcttgagatcaaacatcatcaccatcatcattag

SEQ ID NO: 17 - protein, 430 aa, Artificial Sequence
  EPIL-2-2303407
MYRMQLLSCIALSLALVTNSAPTSSSTKKTQLQLEHLLLDLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSTISTLTGGGGSGGGGSGGGGSGGGGSEVQLLEQSGAELARPGASVKLSCKASGYTFTNYGLSWVKQRPGQVLEWIGEVYPRIGNAYYNEKFKGKATLTADKSSSTASMELRSLTSEDSAVYFCARRGSYDTNYDWYFDVWGQGTTVTVSSGGGGSGGGGSGGGGSELVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKHHHHHH

SEQ ID NO: 18 - DNA, 1293 bp, Artificial Sequence
  EPIL-2-2303407
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagcgctaaatttagctccaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcaccatctcaacactgactggtggtggtggttctggcggcggcggctccggtggtggtggttctggcggcggcggctccgaggtgcagctgctcgagcagtctggagctgagctggcgaggcctggggcttcagtgaagctgtcctgcaaggcttctggctacaccttcacaaactatggtttaagctgggtgaagcagaggcctggacaggtccttgagtggattggagaggtttatcctagaattggtaatgcttactacaatgagaagttcaagggcaaggccacactgactgcagacaaatcctccagcacagcgtccatggagctccgcagcctgacctctgaggactctgcggtctatttctgtgcaagacggggatcctacgatactaactacgactggtacttcgatgtctggggccaagggaccacggtcaccgtctcctcaggtggtggtggttctggcggcggcggctccggtggtggtggttctgagctcgtgatgacccagactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagcttgagatcaaacatcatcaccatcatcattag

SEQ ID NO: 19 - DNA, 4365 bp, Artificial Sequence
  pTT5
gtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacggatctctagcgaattccctctagagggcccgtttctgctagcaagcttgctagcggccgctcgaggccggcaaggccggatcccccgacctcgacctctggctaataaaggaaatttattttcattgcaatagtgtgttggaattttttgtgtctctcactcggaaggacatatgggagggcaaatcatttggtcgagatccctcggagatctctagctagaggatcgatccccgccccggacgaactaaacctgactacgacatctctgccccttcttcgcggggcagtgcatgtaatcccttcagttggttggtacaacttgccaactgaaccctaaacgggtagcatatgcttcccgggtagtagtatatactatccagactaaccctaattcaatagcatatgttacccaacgggaagcatatgctatcgaattagggttagtaaaagggtcctaaggaacagcgatgtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccg